(19) United States
(12) Patent Application Publication
(10) Pub. No.: US 2024/0327326 A1
(43) Pub. Date: Oct. 3, 2024

(54) METHOD FOR PREPARING PT-BASED ALLOY / MOFS CATALYST WITH HIGH HYDROGENATION SELECTIVITY AND APPLICATION THEREOF

(71) Applicant: SUN YAT-SEN UNIVERSITY, Guangdong (CN)

(72) Inventors: Guangqin LI, Guangdong (CN); QINGLIN LIU, Guangdong (CN)

(21) Appl. No.: 17/619,922

(22) PCT Filed: Nov. 17, 2021
(86) PCT No.: PCT/CN2021/131216

§ 371 Date: Feb. 5, 2024

(30) Foreign Application Priority Data
Apr. 6, 2021 (CN) 202110367221.2

(51) Int. Cl. C07C 45/62; B01J 23/89; B01J 31/18; B01J 37/00; B01J 37/02; B01J 37/03; B01J 37/04; B01J 37/06; B01J 37/34; C07C 29/141; C07C 201/12; C07C 209/36

(52) U.S. Cl. CPC C07C 45/62; B01J 23/8906; B01J 23/8913; B01J 23/892; B01J 31/18; B01J 37/009; B01J 37/0236; B01J 37/035; B01J 37/04; B01J 37/06; B01J 37/343; C07C 29/141; C07C 201/12; C07C 209/36; B01J 2231/643; B01J 2231/645; B01J 2231/763; B01J 2531/828; B01J 2531/842; B01J 2531/845; B01J 2531/847; C07C 2601/16

(57) ABSTRACT

The present disclosure relates to the technical field of molecular biology, and in particular to a method for preparing a Pt-based alloy/MOFs catalyst with high hydrogenation selectivity, and a preparation method thereof. The present disclosure prepares a Pt-based alloy/MOFs structure with Pt alloy particles uniformly supported on the surface of MOFs in one step through a simple solvothermal method, the preparation method of the present disclosure is simple, the reaction environment is not harsh and does not require a special atmosphere. The resulting product has a unique structure, with small metal particles, uniform distribution and not easy to lose, and it will not affect the catalytic activity of the metal. In terms of catalytic performance, the obtained Pt alloy/MOFs catalyst can catalytically hydrogenate cinnamaldehyde under normal temperature and pressure, and has excellent performance. In addition, the catalyst can also catalyze the selective hydrogenation of 3-nitrostyrene, catalyze the dehydrogenation of tetrahydroquinoline, which proves that the catalyst of the present disclosure has a wide range of applications.

This patent application claims the benefit and priority of Chinese Patent Application No. 202110367221.2 filed on Apr. 6, 2021, the disclosure of which is incorporated by reference herein in its entirety as part of the present application.

### TECHNICAL FIELD

The present disclosure relates to the technical field of molecular biology, and in particular to a method for preparing a Pt-based alloy/MOFs catalyst with high hydrogenation selectivity, and a preparation method thereof.

### BACKGROUND ART

In recent years, selective hydrogenation of α, β-unsaturated aldehydes to obtain a single product has become a research hotspot, because both hydrogenation products of C═C double bond and hydrogenation products of C═O double bond can be used in the fields of reaction intermediates, perfumes, food processing, etc. Since C═O double bonds are thermodynamically more stable than C═C double bonds, it is still difficult to hydrogenate C═O bonds with high selectivity to obtain unsaturated alcohols. In addition, there are some selective hydrogenation reactions such as the selective formation of 3-aminostyrene from 3-nitrostyrene, which is also very important but difficult to achieve high selectivity. Pt-based precious metals and the alloys thereof are often used for selective catalytic hydrogenation. When elements such as Fe and Co are added, Pt-based alloys exhibit higher selectivity for hydrogenation of C═O double bonds. However, Pt-based precious metals are prone to agglomeration during synthesis or reaction, resulting in a gradual decrease in catalytic performance. Therefore, it is usually necessary to compound the precious metal with a stable carrier to stabilize the performance of the catalyst.

Metal organic frameworks (MOFs) are widely used in heterogeneous catalysis due to their large specific surface area, high porosity, strong stability, etc., and when precious metals are combined with MOFs, due to the overflow effect of hydrogen, the rate of hydrogenation reaction can be accelerated. At present, there have been many reports on the use of Pt-based nanoparticles and MOFs composites for selective hydrogenation. The most classic is MOFs@Pt@MIL-101(Fe), the preparation method of the Pt-based/MOFs composite is as follows: first synthesizing Pt nanoparticles and MOFs carrier separately, mixing the two, then adding a mixture of ligand and Fe salt, and finally heating at 120° C. The Pt-based/MOFs composite has a sandwich structure, which has higher cinnamyl alcohol selectivity and cycle stability than the non-sandwich structure of MOFs@Pt. Through subsequent characterization and theoretical calculations, it is found that positively charged Pt is more likely to adsorb the C═O double bond in cinnamaldehyde, MIL-101(Fe) also has a similar effect, the two together promote the selective hydrogenation of cinnamaldehyde to cinnamyl alcohol, thereby accelerating the rate of hydrogenation reaction.

In addition, the metal alloy PtFe has also been reported to be used in selective hydrogenation. Studies have shown that when PtFe nanowires are used in the selective hydrogenation of α, β-unsaturated aldehydes, they have good catalytic activity and hydrogenation selectivity of C═O double bond, which is better than pure Pt nanowires. However, the morphology of PtFe nanowires will be destroyed after multiple cycles of reaction, resulting in a gradual decline in their catalytic performance.

At present, although considerable research progress has been made in selective hydrogenation catalysts, the existing selective hydrogenation catalysts still have problems such as multiple synthesis steps and complexity in their preparation methods, and the finally obtained catalyst still needs to catalyze hydrogenation under relatively severe reaction conditions. At the same time, the preparation of most existing selective hydrogenation catalysts is first to synthesize the precious metal or alloy nanoparticles, and then load the particles on the carrier through mechanical stirring. Although the resulting catalyst has good catalytic activity and hydrogenation selectivity, since the metal or alloy nanoparticles are exposed to the outside, and the metal or alloy and the carrier are only physically adsorbed without interaction, they are easy to fall, agglomerate or deform during the catalytic process, so it is difficult to guarantee their own cycle. In addition, some non-precious metal salts are more difficult to be reduced, and require intense reaction temperature or special gas atmospheres (such as CO, H₂, etc.), which severely limits their large-scale synthesis.

### SUMMARY

In order to overcome the above shortcomings of the prior art, the present disclosure proposes a method for preparing a Pt-based alloy/MOFs catalyst with high hydrogenation selectivity, which cleverly uses the organic ligand terephthalic acid (BDC) to promote the co-reduction of platinum acetylacetonate and acetylacetonate metal salt, which makes the Pt alloy uniformly distributed on the surface of the MOFs carrier. The structure of Pt alloy/MOFs is synthesized by one-step method, which is not only suitable for preparing catalysts with different combinations of Pt alloys, but also for preparing Pt alloy catalysts with different carriers. This type of catalyst is used in the selective catalytic hydrogenation of α, β-unsaturated aldehydes, and has excellent catalytic activity and unsaturated alcohol selectivity; At the same time, it can also selectively catalyze the hydrogenation of 3-nitrostyrene to obtain 3-aminostyrene with high yield, and has good cycle performance. In addition, the catalyst also exhibits excellent catalytic dehydrogenation capacity and atmospheric hydrogen storage capacity.

In order to achieve the above objective, the technical scheme adopted by the present disclosure is as follows:

The primary objective of the present disclosure is to provide a method for preparing a Pt-based alloy/MOFs catalyst, specifically: adding a MOFs carrier to DMF (dimethylformamide), dispersing and stirring at room temperature, then adding platinum acetylacetonate and terephthalic acid, continuing to stir at room temperature, adding a certain amount of acetylacetone metal salt, then stirring at room temperature, placing the resulting solution at 140-160° C. for continuous stirring for 10-15 h, and then performing centrifugation, washing and drying to obtain the Pt-based alloy/MOFs catalyst.

In some embodiments, the present disclosure is suitable for different MOFs carriers, the MOFs carrier includes UiO-66-NH₂, UiO-66 or MIL-101(Cr). Further, the MOFs carrier is UiO-66-NH₂.

In some embodiments, the present disclosure is suitable for reducing different non-precious metal salts, and is suitable for preparing catalysts of various combinations of different Pt alloys, the acetylacetone metal salt includes ferrous acetylacetonate, nickel acetylacetonate, or cobalt acetylacetonate. Further, the acetylacetone metal salt is ferrous acetylacetonate.

The present disclosure synthesizes the structure of the Pt alloy/MOFs carrier in one step through a simple solvothermal method, and utilizes the effect of terephthalic acid to promote the co-reduction of platinum acetylacetonate and the acetylacetone metal salt to make the Pt alloy uniformly distributed on the MOFs carrier, and the size of the Pt alloy particles is small (nano-level), which eliminates the complicated steps of first synthesizing the alloy and then loading. It is not only suitable for the preparation of catalysts with different combinations of Pt alloys, but also for the preparation of Pt alloy catalysts with different carriers. At the same time, the obtained Pt alloy/MOFs is used to the catalytic hydrogenation of cinnamaldehyde, the reaction can be carried out under normal temperature and pressure, the reaction conditions are mild, the reactivity and cinnamyl alcohol selectivity of Pt alloy/MOFs is better than pure Pt/UiO-66-NH₂, and it also exhibits good recyclability, which proves that the synthesized Pt alloy/MOFs catalyst of the present disclosure has superior performance for the selective hydrogenation of α, β-unsaturated aldehydes, good catalytic activity, unsaturated alcohol selectivity and reaction activity stability. Wherein, the performance of PtFe₂/UiO-66-NH₂ catalyst is particularly excellent, especially PtFe₂/UiO-66-NH₂ is the most prominent. PtFe₂/UiO-66-NH₂ catalyst can catalyze the hydrogenation of cinnamaldehyde under normal temperature and pressure. It can achieve 98.9% conversion and 95.4% cinnamyl alcohol selectivity after 12 h, and there is no obvious performance degradation after 5 cycles of reaction. In addition, using PtFe₂/UiO-66-NH₂ catalyst to catalyze 3-nitrostyrene can also obtain 3-aminostyrene with a high yield. PtFe₂/UiO-66-NH₂ catalyst can catalyze hydrogenation of 3-nitrostyrene under normal temperature and pressure, it can reach 96.1% conversion and 92.3% 3-aminostyrene selectivity in 2 h, and the performance remains good after 5 cycles of reaction, which further proves that the Pt alloy/MOFs catalyst of the present disclosure can be applied to multiple selective hydrogenation system. The catalyst not only has high-efficiency selective hydrogenation performance, but also has good application prospects in hydrogen storage and dehydrogenation. The PtFe₂/UiO-66-NH₂ catalyst can catalyze the dehydrogenation of tetrahydroquinoline to quinoline, and its catalytic ability is better than that of the single metal catalyst Pt/UiO-66-NH₂. In addition, PtFe₂/UiO-66-NH₂ can store hydrogen under normal pressure, and the hydrogen storage capacity can reach 7.4 mmol H₂/g_{cat}.

The present disclosure has found through various characterizations that in the PtFe/MOFs structure, the ratio of Pt and Fe elements is basically equivalent to the feed ratio, which proves that the preparation method of the Pt-based alloy/MOFs catalyst of the present disclosure can also accurately control the ratio of Pt and Fe, and the optimal selective hydrogenation performance can be obtained by adjusting the appropriate ratio of Pt and Fe.

The methods for synthesizing Pt alloys in the prior art need to be carried out under relatively severe conditions (such as in a CO atmosphere or heating above 200° C., etc.), which is dangerous, and is not energy-saving and environmentally friendly. The DMF used in the present disclosure is both a solvent and a reducing agent, and the reaction conditions are relatively mild.

The Pt alloy synthesized in the prior art does not have good anchoring points, the obtained particles are relatively large, the catalytic activity is weak, and it is easy to fall off or deform during the catalytic process. The one-step solvothermal method of the present disclosure utilizes organic ligand terephthalic acid (BDC) to promote the co-reduction of platinum acetylacetonate and acetylacetonate metal salt, so that the synthesized platinum alloy particles are smaller in size and uniformly dispersed in MOFs surface; Moreover, the promotion effect of the organic ligand terephthalic acid can also enhance the interaction between the alloy particles and the MOFs carrier, so that the resulting structure can not only expand the contact area of the active catalytic site, increase the catalytic activity, but also improve the stability of the catalyst. In addition, when using UiO-66-NH₂, a kind of MOF with NH₂ group, as the carrier, the NH₂ in MOFs can immobilize the metal salt, and the lone pair of electrons on NH₂ will promote the adsorption of the C═O double bond by the substrate, thereby promoting the catalytic activity while further enhancing the selectivity to the C═O double bond.

Further, the method for preparing UiO-66-NH₂ is as follows: adding zirconium tetrachloride and 2-aminoterephthalic acid to DMF, then adding acetic acid, stirring at room temperature, reacting under high temperature of 110-130° C. and high pressure for 10-15 h, and finally performing centrifugation, washing and drying to obtain the UiO-66-NH₂.

Specifically, the solid-to-liquid ratio of 2-aminoterephthalic acid to DMF is 39.5 mg/50 mL.

Further, the preparation method of the UiO-66 is as follows: adding zirconium tetrachloride and terephthalic acid to DMF, then adding acetic acid, stirring at room temperature, reacting under high temperature of 110-130° C. and high pressure for 10-15 h, and finally performing centrifugation, washing and drying to obtain the UiO-66.

Specifically, the solid-to-liquid ratio of terephthalic acid to DMF is 36 mg/50 mL.

Specifically, in the preparation process of UiO-66-NH₂ and UiO-66, the solid-to-liquid ratio of zirconium tetrachloride to DMF is 52 mg/50 mL.

Specifically, in the preparation process of UiO-66-NH₂ and UiO-66, the volume ratio of acetic acid to DMF is 3:25.

Specifically, in the preparation process of the UiO-66-NH₂ and UiO-66, the temperature of reacting under high temperature and high pressure is 120° C., and the time is 12 h.

In some embodiments, in the preparation process of the Pt-based alloy/MOFs catalyst, UiO-66-NH₂ and UiO-66, the washing is washing twice with DMF and ethanol respectively; and the drying is vacuum drying at 120° C.

In some embodiments, the solid-to-liquid ratio of the MOFs carrier to DMF is (40-60) mg/5 mL. Further, the solid-to-liquid ratio of the MOFs carrier to DMF is 50 mg/5 mL.

In some embodiments, the ratio of the amount-of-substance of platinum acetylacetonate to the volume of DMF is 0.01 mmol:5 mL, and the solid-to-liquid ratio of terephthalic acid to DMF is (16-20) mg/5 mL. Further, the solid-to-liquid ratio of terephthalic acid to DMF is 18 mg/5 mL.

In some embodiments, the ratio of the amount-of-substance of the acetylacetone metal salt to the volume of DMF is (0.01-0.03) mmol:5 mL. Further, the ratio of the amount-of-substance of the acetylacetone metal salt to the volume of DMF is 0.02 mmol:5 mL.

The second object of the present disclosure is to provide a Pt-based alloy/MOFs catalyst prepared by the above preparation method.

In some embodiments, the Pt-based alloy/MOFs catalyst is PtFe₁/UiO-66-NH₂, PtFe₂/UiO-66-NH₂ or PtFe₃/UiO-66-NH₂, PtM₂/UiO-66-NH₂, PtFe₂/UiO-66 or PtFe₂/MIL-101(Cr); where M in the PtM₂/UiO-66-NH₂ is Ni or Co.

The third object of the present disclosure is to provide the application of the above Pt-based alloy/MOFs catalyst in selective hydrogenation reactions.

In some embodiments, the selective hydrogenation reaction includes the selective hydrogenation reaction of α, β-unsaturated aldehyde and the selective hydrogenation reaction of 3-nitrostyrene.

Further, the selective hydrogenation reaction of α, β-unsaturated aldehyde is a hydrogenation reaction of cinnamaldehyde.

In some embodiments, the catalytic conditions for the hydrogenation reaction of cinnamaldehyde are as follows: adding the Pt-based alloy/MOFs catalyst and cinnamaldehyde to the mixture of 2.5 mL of water and 2.5 mL of isopropanol with the added amount of Pt-based alloy/MOFs catalyst of 5 mg and the added amount of cinnamaldehyde of 0.3 mmol, performing ultrasonic treatment for 10 min to obtain a uniform mixture, transferring the mixture to a 25 mL reaction flask, putting on a rubber stopper, performing vacuuming with a vacuum pump for 1 min, then quickly plunging into a self-made hydrogen balloon to make the hydrogen pressure consistent with the atmospheric pressure, and stirring for 12 h at room temperature.

In some embodiments, the conditions for the selective hydrogenation of 3-nitrostyrene are as follows: adding the Pt-based alloy/MOFs catalyst and 3-nitrostyrene to 5 mL of ethanol with the added amount of Pt-based alloy/MOFs catalyst of 5 mg and the added amount of 3-nitrostyrene of 0.3 mmol, performing ultrasonic treatment for 5 min to obtain a uniform mixture, transferring the mixture to a 25 mL reaction flask, putting on a rubber stopper, performing vacuuming with a vacuum pump for 1 min, then quickly plunging into a self-made hydrogen balloon to make the hydrogen pressure consistent with the atmospheric pressure, and stirring for 5 h at room temperature.

Compared with the prior art, the beneficial effects of the present disclosure are as follows:

The present disclosure provides a method for preparing a Pt-based alloy/MOFs catalyst, which can obtain Pt-based alloy/MOFs structure with Pt alloy particles uniformly supported on the surface of MOFs in one step through a simple solvothermal method, the preparation method of the present disclosure is simple, and is not only suitable for the preparation of catalysts with different combinations of Pt alloys, but also for the preparation of Pt alloy catalysts with different carriers. Moreover, the reaction environment is not harsh and does not require a special atmosphere. The resulting product has a unique structure, with small metal particles, uniform distribution and not easy to lose, and it will not affect the catalytic activity of the metal. In terms of catalytic performance, the obtained Pt alloy/MOFs catalyst can catalytically hydrogenate cinnamaldehyde under normal temperature and pressure, and has excellent performance. In addition, the catalyst can also catalyze the selective hydrogenation of 3-nitrostyrene, catalyze the dehydrogenation of tetrahydroquinoline, and improve the hydrogen storage under normal pressure, which proves that the catalyst of the present disclosure has a wide range of applications. In addition to the highly efficient selective hydrogenation performance, the catalyst of the present disclosure also has good application prospects in the fields of hydrogen storage, hydrogenation and dehydrogenation, etc.

### BRIEF DESCRIPTION OF THE DRAWINGS

FIG. 1 is a field emission scanning electron microscope image of UiO-66-NH₂;

FIG. 2 is a TEM image of PtFe₁/UiO-66-NH₂;

FIG. 3 is a TEM image of PtFe₂/UiO-66-NH₂;

FIG. 4 is a TEM image of PtFe₃/UiO-66-NH₂;

FIG. 5 is a TEM image of PtFe₂/UiO-66-NH₂;

FIG. 6 is an element linear scan diagram of PtFe₂/UiO-66-NH₂;

FIG. 7 is an element mapping diagram of PtFe₂/UiO-66-NH₂;

FIG. 8 is a TEM image of PtNi₂/UiO-66-NH₂;

FIG. 9 is a TEM image of PtCo₂/UiO-66-NH₂;

FIG. 10 is a TEM image of PtFe₂/UiO-66;

FIG. 11 is a TEM image of PtFe₂/MIL-101(Cr);

FIG. 12 is a TEM image of Pt/UiO-66-NH₂ synthesized without terephthalic acid;

FIG. 13 is a TEM image of Pt/UiO-66-NH₂ synthesized with the addition of terephthalic acid;

FIG. 14 is a TEM image of PtFe₂/UiO-66-NH₂ synthesized without terephthalic acid;

FIG. 15 is an XRD pattern of various hydrogenation catalysts;

FIG. 16 is a schematic diagram of the hydrogenation of cinnamaldehyde;

FIG. 17 shows the conversion and selectivity of the hydrogenation reaction of cinnamaldehyde catalyzed by Pt/UiO-66-NH₂ over time;

FIG. 18 shows the conversion and selectivity of the hydrogenation reaction of cinnamaldehyde catalyzed by PtFe₁/UiO-66-NH₂ over time;

FIG. 19 shows the conversion and selectivity of the hydrogenation reaction of cinnamaldehyde catalyzed by PtFe₂/UiO-66-NH₂ over time;

FIG. 20 shows the conversion and selectivity of the hydrogenation reaction of cinnamaldehyde catalyzed by PtFe₃/UiO-66-NH₂ over time;

FIG. 21 shows the conversion of cinnamaldehyde and selectivity of cinnamyl alcohol catalyzed by PtFe₂/UiO-66-NH₂ in multiple cycles of hydrogenation;

FIG. 22 is a schematic diagram of the hydrogenation of 3-nitrostyrene;

FIG. 23 shows the conversion and selectivity of the hydrogenation reaction of 3-nitrostyrene catalyzed by Pt/UiO-66-NH₂ over time;

FIG. 24 shows the conversion and selectivity of the hydrogenation of 3-nitrostyrene catalyzed by PtFe₂/UiO-66-NH₂ over time;

FIG. 25 shows the conversion of 3-nitrostyrene and the selectivity of 3-aminostyrene catalyzed by PtFe₂/UiO-66-NH₂ in multiple cycles of hydrogenation.

FIG. 26 is a graph showing the performance of Pt/UiO-66-NH₂ and PtFe₂/UiO-66-NH₂ in catalyzing the dehydrogenation of tetrahydroquinoline to quinoline.

FIG. 27 shows the hydrogen adsorption isotherm curve of PtFe₂/UiO-66-NH₂ (1 bar, 77 K).

### DETAILED DESCRIPTION OF THE EMBODIMENTS

The present disclosure will be further described below in conjunction with embodiments and drawings.

The specific embodiments of the present disclosure will be further described below. It should be noted here that the description of these embodiments is used to help understand the present disclosure, but does not constitute a limitation to the present disclosure. In addition, the technical features involved in the various embodiments of the present disclosure described below can be combined with each other as long as they do not conflict with each other.

The experimental methods in the following examples, unless otherwise specified, are all conventional methods, and the test materials used in the following examples, unless otherwise specified, are all commercially available through conventional commercial channels.

### Example 1: Preparation of PtFe/UiO-66-NH₂ Catalyst

(1) Preparation of UiO-66-NH₂: 52 mg of zirconium tetrachloride and 39.5 mg of 2-aminoterephthalic acid were added to 50 mL of DMF, then 6 mL of acetic acid was added thereto, the mixture was stirred at room temperature for 30 min, the resulting mixture was transferred to the liner of a reactor, then the liner of the polytetrafluoroethylene reactor was placed into a high pressure reactor, and the mixture was reacted under high temperature of 120° C. and high pressure for 12 h. The resulting mixture was centrifuged at a speed of 8000 rpm, the supernatant was removed, the remaining was washed twice with DMF and ethanol respectively, and finally subjected to vacuum drying at 120° C. to obtain UiO-66-NH₂.

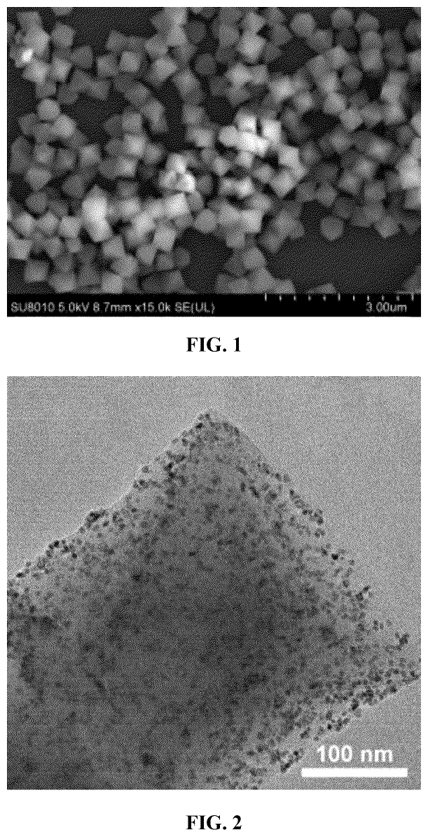

The prepared UiO-66-NH₂ was observed with a scanning electron microscope (Hitachi SU8010). As shown in FIG. 1, the MOF has an octahedral structure with uniform size.

(2) Preparation of PtFe/UiO-66-NH₂: 50 mg of UiO-66-NH₂ was added to 5 mL of DMF, the mixture was subjected to ultrasonic treatment to uniform dispersion (power of 360 W, the time of ultrasonic treatment of 5 min), and then stirred at room temperature for 30 min to obtain a solution A; then 0.01 mmol of platinum acetylacetonate and 18 mg of terephthalic acid were added to the solution A, and stirred at room temperature for 10 min to obtain an uniform solution B; then a certain amount (0.01 mmol, 0.02 mmol, 0.03 mmol) of ferrous acetylacetonate was added to the solution B, the mixture was continued to stir at room temperature for 30 min, the resulting solution was continued to stir at 150° C. for 12 h at a stirring speed of 600 rpm, then the resulting mixture was centrifuged at a speed of 8000 rpm, the supernatant was removed, the remaining was washed twice with DMF and ethanol respectively, and finally dried under vacuum at 60° C. overnight to obtain PtFe/UiO-66-NH₂.

Wherein, the product with 0.01 mmol of iron acetylacetonate added is named PtFe₁/UiO-66-NH₂, the product with 0.02 mmol of iron acetylacetonate added is named PtFe₂/UiO-66-NH₂, and the product with 0.03 mmol of iron acetylacetonate added is named PtFe₃/UiO-66-NH₂.

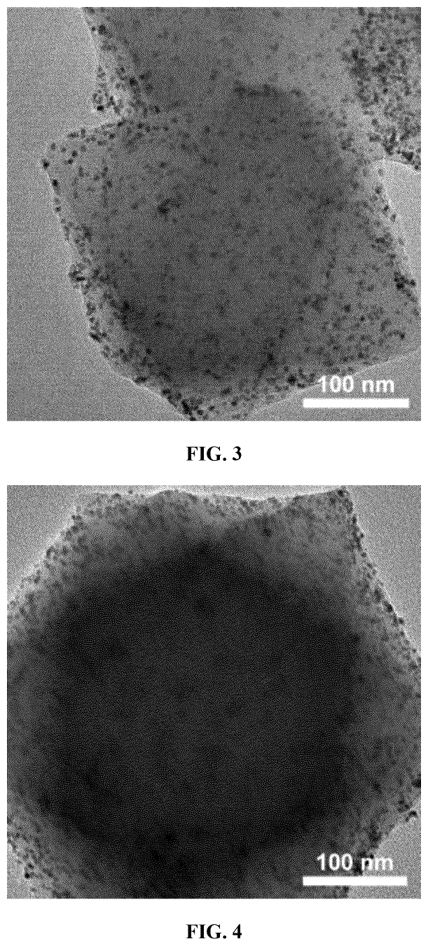

The PtFe₁/UiO-66-NH₂, PtFe₂/UiO-66-NH₂ and PtFe₃/UiO-66-NH₂ prepared above were observed by transmission electron microscope (JEM-1400 Plus, JEOL). As can be seen from the transmission electron micrographs in FIG. 2-4, a large number of particles with very small diameters are uniformly distributed on the surface of UiO-66-NH₂, which proves that the added terephthalic acid can promote the reduction of Pt salts and Fe salts, and is beneficial to the dispersion of the particles. At the same time, it can be seen that adding different amounts of iron acetylacetonate does not destroy the morphology of the MOF, and the size of the metal particles obtained is not very different.

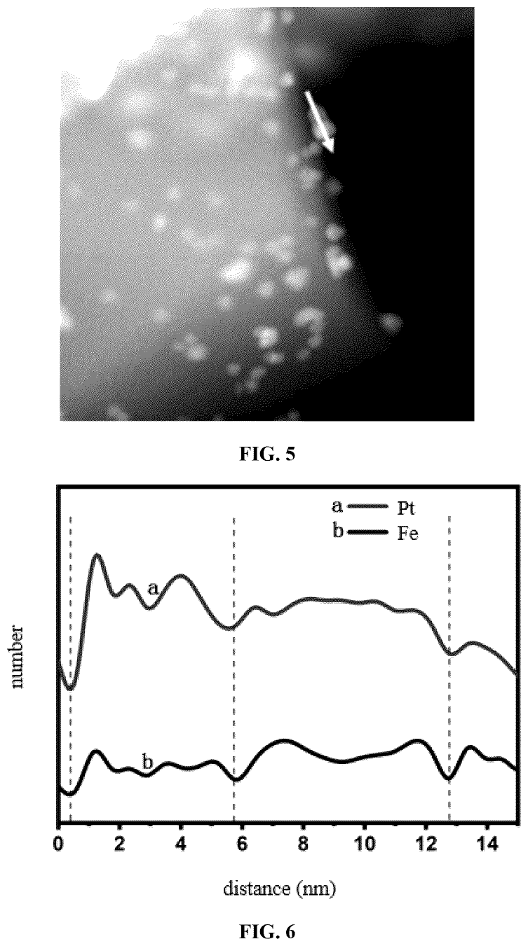

The PtFe₂/UiO-66-NH₂ prepared above was observed with high-angle annular dark-field scanning TEM (ARM200F, JEOL). As shown in FIG. 5, the brighter particles in the figure are PtFe alloys. The element linear scan analysis of the two PtFe particles pointed by the white arrow shows (FIG. 6) that the distribution trends of Pt and Fe are basically the same, which proves that Pt and Fe form an alloy.

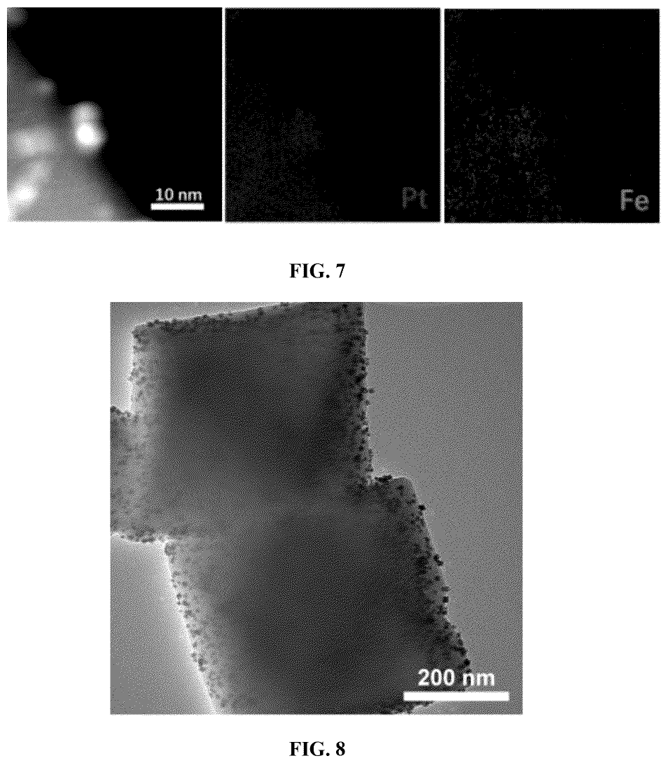

The PtFe₂/UiO-66-NH₂ prepared above was subjected to element mapping analysis, and the result is shown in FIG. 7. The left image in the figure is the ring-shaped dark field image of the sample, in which the bright place is metal, and the dark place is MOF; The two images on the right are based on the element distribution of the image on the left. It can be proved that there are more Pt and Fe at the same time in the bright place, and the element distribution positions of Pt and Fe are basically the same, which also proves that Pt and Fe form an alloy.

### Example 2: Preparation of PtM₂/UiO-66-NH₂ (M=Ni or Co) Catalyst

(1) The preparation of UiO-66-NH₂ is the same as in Example 1.

(2) Preparation of PtM₂/UiO-66-NH₂ (M=Ni or Co): 50 mg of UiO-66-NH₂ was added to 5 mL of DMF, the mixture was subjected to ultrasonic treatment to uniform dispersion (power of 360 W, the time of ultrasonic treatment of 5 min), and then stirred at room temperature for 30 min to obtain a solution A; then 0.01 mmol of platinum acetylacetonate and 18 mg of terephthalic acid were added to the solution A, and stirred at room temperature for 10 min to obtain an uniform solution B; then 0.02 mmol of nickel acetylacetonate or cobalt acetylacetonate was added to the solution B, the mixture was continued to stir at room temperature for 30 min, the resulting solution was continued to stir at 150° C. for 12 h at a stirring speed of 600 rpm, then the mixture was centrifuged at a speed of 8000 rpm, the supernatant was removed, the remaining was washed twice with DMF and ethanol respectively, and finally dried under vacuum at 60° C. overnight to obtain PtNi₂/UiO-66-NH₂ and PtCo₂/UiO-66-NH₂.

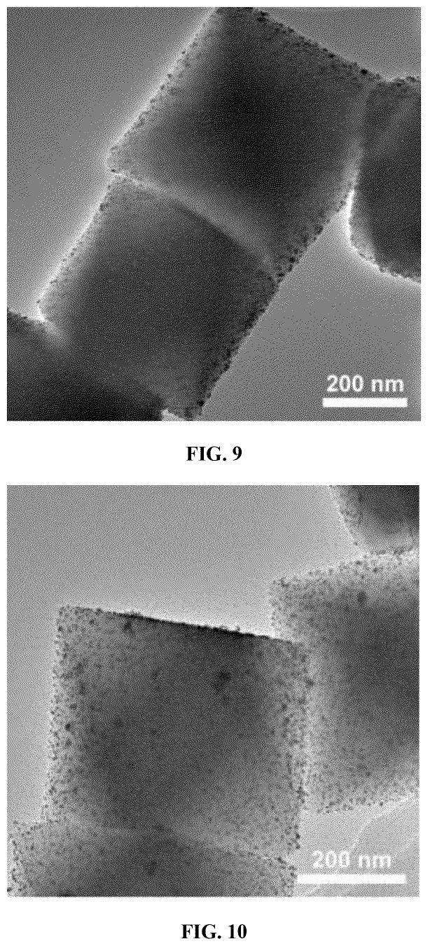

The PtNi₂/UiO-66-NH₂ and PtCo₂/UiO-66-NH₂ prepared above were analyzed by transmission electron microscopy (TEM). It can be seen from FIG. 8 and FIG. 9 that the metal particles are uniformly loaded on the UiO-66-NH₂ surface.

### Example 3: Preparation of PtFe₂/UiO-66 and PtFe₂/MIL-101(Cr) Catalysts

(1) Preparation of UiO-66: 52 mg of zirconium tetrachloride and 36 mg of terephthalic acid were added to 50 mL of DMF, then 6 mL of acetic acid was added thereto, the mixture was stirred at room temperature for 30 min, the mixed solution was transferred to the liner of a polytetrafluoroethylene reactor, then the liner was placed into a high pressure reactor, the temperature was raised to 120° C., the solvent was volatilized to form a high-pressure environment, then the mixture was reacted under high temperature of 120° C. and high pressure for 12 h, the resulting mixture was centrifuged at 8000 rpm, the supernatant was removed, the remaining was washed twice with DMF and absolute ethanol respectively, and finally subjected to vacuum drying at 120° C. to obtain UiO-66.

(2) Preparation of MIL-101(Cr): 266.5 mg of chromium trichloride hexahydrate and 166.1 mg of terephthalic acid were dispersed in 7.2 mL of water, stirred vigorously at room temperature for 3 min, the resulting mixture was transferred to the liner of polytetrafluoroethylene reactor, then the liner was placed into a stainless steel reactor, and the mixture was reacted at 210° C. for 24 h. The temperature was naturally cooled to room temperature, then the obtained mixture was transferred to the centrifuge tube and centrifuged at a speed of 1000 rpm for 3 min, the crystalline terephthalic acid was precipitated, the upper mixture was taken, then centrifuged at 5000 rpm for 10 min, and washed twice with DMF and once with absolute ethanol. The obtained solid was placed in a vacuum drying oven, and dried overnight at 150° C. to obtain MIL-101 (Cr).

(3) Preparation of PtFe₂/MOFs: 50 mg of UiO-66 or MIL-101(Cr) was added to 5 mL of DMF, subjected to ultrasonic treatment to uniform dispersion (power of 360 W, the time of ultrasonic treatment of 5 min), and then stirred at room temperature for 30 min to obtain a solution A; then 0.01 mmol of platinum acetylacetonate and 18 mg of terephthalic acid were added to the solution A, and stirred at room temperature for 10 min to obtain an uniform solution B; then 0.02 mmol of ferrous acetylacetonate was added to the solution B, the mixture was continued to stir at room temperature for 30 min, the resulting solution was continued to stir at 150° C. for 12 h at a stirring speed of 600 rpm, then the mixed solution was centrifuged at a speed of 8000 rpm, the supernatant was removed, the remaining was washed twice with DMF and ethanol respectively, and finally dried under vacuum at 60° C. overnight to obtain PtFe₂/UiO-66 and PtFe₂/MIL-101(Cr).

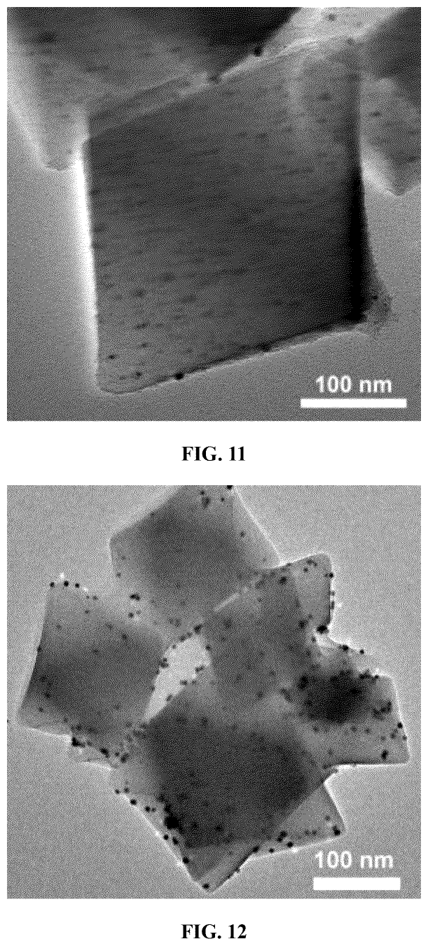

The PtNi₂/UiO-66 and PtFe₂/MIL-101(Cr) prepared above were analyzed by transmission electron microscopy (TEM). It can be seen from FIG. 10 that the metal particles are uniformly loaded on the UiO-66 surface; It can be seen from FIG. 11 that the metal particles are uniformly loaded on the PtFe₂/MIL-101(Cr) surface.

### Comparative Example 1: Preparation of Pt/UiO-66-NH₂Catalyst

(1) The preparation of UiO-66-NH₂ is the same as in Example 1.

(2) Preparation of Pt/UiO-66-NH₂:

Preparation method 1: 50 mg of UiO-66-NH₂ was added to 5 mL of DMF, subjected to ultrasonic treatment to uniform dispersion, and then stirred at room temperature for 30 min to obtain a solution A; then 0.01 mmol of platinum acetylacetonate and 18 mg of terephthalic acid were added to the solution A, and stirred at room temperature for 30 min, the resulting solution was continued to stir at 150° C. for 12 h at a stirring speed of 600 rpm, then the mixed solution was centrifuged at a speed of 8000 rpm, the supernatant was removed, the remaining was washed twice with DMF and ethanol respectively, and finally dried under vacuum at 60° C. overnight to obtain Pt/UiO-66-NH₂.

Preparation method 2: The specific method is the same as the preparation method 1, except that terephthalic acid is not added during the preparation process.

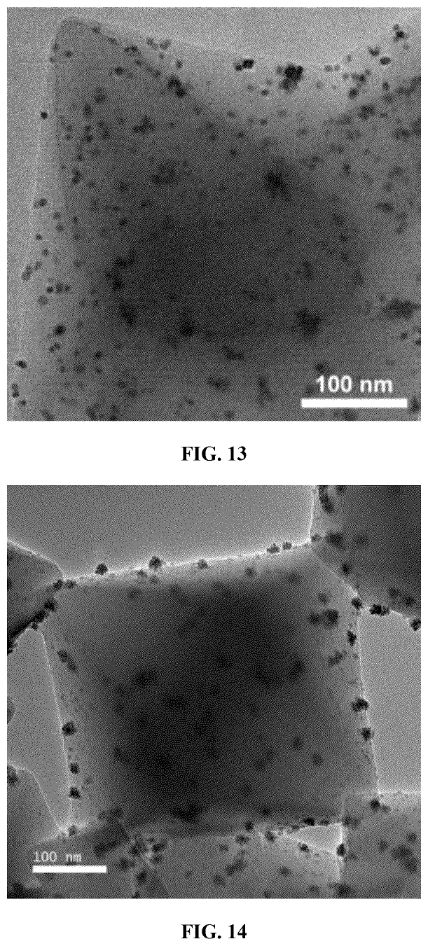

The Pt/UiO-66-NH₂ prepared with and without terephthalic acid was observed by transmission electron microscope (JEM-1400 Plus, JEOL). From the transmission electron microscope image of Pt/UiO-66-NH₂ prepared without adding terephthalic acid in FIG. 12, the octahedral structure can also be observed, the size of UiO-66-NH₂ is consistent with the SEM image, but the surface of UiO-66-NH₂ has only relatively sparse metal particles, indicating that the Pt salt has not been well reduced. From the transmission electron microscope image of Pt/UiO-66-NH₂ prepared with terephthalic acid in FIG. 13, it can be seen that a large number of particles with a small particle size are uniformly distributed on the surface of UiO-66-NH₂, which proves that the added terephthalic acid can promote the reduction of Pt salt, and it is conducive to particle dispersion.

### Comparative Example 2: Preparation of PtFe₂/UiO-66-NH₂ Catalyst without Adding Terephthalic Acid

The specific method is the same as in Example 1, except that terephthalic acid is not added during the preparation process.

The PtFe₂/UiO-66-NH₂ prepared without terephthalic acid was observed by transmission electron microscope (JEM-1400 Plus, JEOL). It can be seen from FIG. 14 that there are larger particles and sparse metal particles on the surface of UiO-66-NH₂, indicating that the Pt salt and Fe salt have not been well reduced.

### Comparative Example 3: Preparation of Pt/UiO-66 Catalyst

(1) The preparation of UiO-66 is the same as in Example 3.

(2) Preparation of Pt/UiO-66: 50 mg of UiO-66 was added to 5 mL of DMF, subjected to ultrasonic treatment to uniform dispersion, and then stirred at room temperature for 30 min to obtain a solution A; then 0.01 mmol of platinum acetylacetonate and 18 mg of terephthalic acid were added to the solution A, and stirred at room temperature for 30 min, the resulting solution was continued to stir at 150° C. for 12 h at a stirring speed of 600 rpm, then the mixed solution was centrifuged at a speed of 8000 rpm, the supernatant was removed, the remaining was washed twice with DMF and ethanol respectively, and finally dried under vacuum at 60° C. overnight to obtain Pt/UiO-66.

### Experimental Example 1: Mass Percentage of Pt and Molar Ratio of Pt to Fe Analysis

The mass percentage of Pt and molar ratio of Pt to Fe of heterogeneous catalysts (PtFe₁/UiO-66-NH₂, PtFe₂/UiO-66-NH₂, PtFe₃/UiO-66-NH₂, PtFe₂/UiO-66, PtFe₂/MIL-101 (Cr), and Pt/UiO-66-NH₂) were analyzed.

As shown in Table 1, the Pt content of the various catalysts is roughly the same, which proves that the change of synthesis conditions will not affect the reduction of Pt. The actual ratio of Pt and Fe is equivalent to the feed ratio, which proves that the method of the present disclosure can accurately adjust the ratio of bimetals to obtain a catalyst with the best performance.

TABLE 1
Mass percentage of Pt and molar ratio of Pt
to Fe of various heterogeneous catalysts

| | | mass percentage of | molar ratio of Pt to |
|---|---|---|---|
| No. | catalyst | Pt/wt % | Fe |
| 1 | Pt/UiO-66-NH₂ | 2.77 | — |
| 2 | PtFe₁/UiO-66-NH₂ | 2.64 | 0.91 |
| 3 | PtFe₂/UiO-66-NH₂ | 2.47 | 0.42 |
| 4 | PtFe₃/UiO-66-NH₂ | 2.33 | 0.23 |
| 5 | PtFe₂/UiO-66 | 2.76 | 0.38 |
| 6 | PtFe₂/MIL-101 (Cr) | 2.53 | 0.45 |

### Experimental Example 2: X-Ray Powder Diffraction

The PtFe₁/UiO-66-NH₂, PtFe₂/UiO-66-NH₂ and PtFe₃/UiO-66-NH₂ of Example 1, the PtFe₂/UiO-66 of Example 3, and the Pt/UiO-66-NH₂ of Comparative Example 1, Pt/UiO-66, UiO-66-NH₂ and UiO-66 of Comparative Example 3 were analyzed by X-ray powder diffraction pattern (XRD) using Rigaku's SmartLab instrument. It can be seen from FIG. 15 that the XRD of all samples retain the peaks of the original carrier UiO-66 or UiO-66-NH₂, which proves that this solvothermal method will not damage the crystal form and structure of MOF. In addition, from the enlarged image on the right, it can be seen that there is no peak at around 40 degrees, which proves that no larger metal particles are formed.

### Experimental Example 3: Activity and Selectivity Analysis of Selective Catalytic Hydrogenation of Cinnamaldehyde

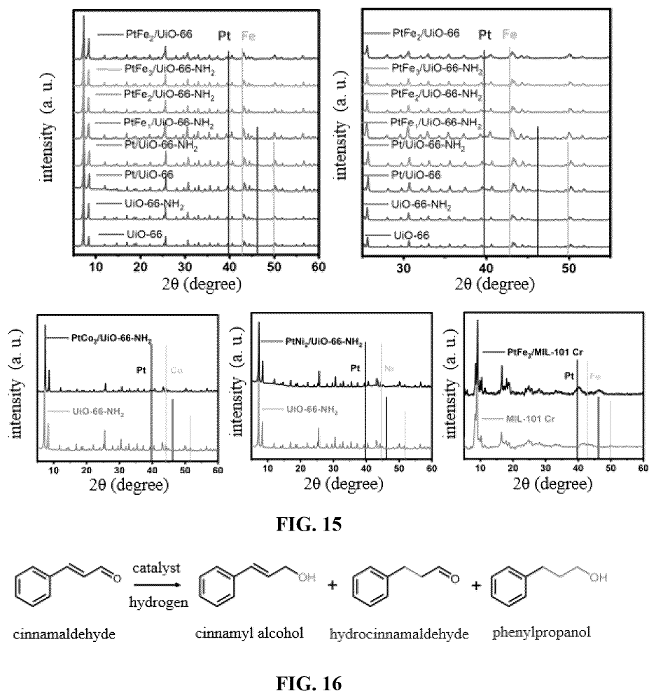

Using a typical α, β-unsaturated aldehyde-cinnamaldehyde as the catalytic substrate, and the catalytic activity and product selectivity of different catalysts (PtFe₁/UiO-66-NH₂, PtFe₂/UiO-66-NH₂, PtFe₃/UiO-66-NH₂, PtCo₂/UiO-66-NH₂, PtNi₂/UiO-66-NH₂, PtFe₂/MIL-101 (Cr), PtFe₂/UiO-66, Pt/UiO-66-NH₂, Pt/UiO-66, UiO-66-NH₂) were compared. The principle of the hydrogenation of cinnamaldehyde is shown in FIG. 16. Cinnamicaldehyde is hydrogenated into cinnamyl alcohol, hydrocinnamaldehyde and phenylpropanol under the action of catalyst and hydrogen. The ideal product is cinnamyl alcohol.

The reaction conditions of heterogeneous catalytic hydrogenation are as follows: the catalyst and cinnamaldehyde were added to the mixture of 2.5 mL of water and 2.5 mL of isopropanol with the added amount of catalyst of 5 mg and the added amount of cinnamaldehyde of 0.3 mmol, the resulting mixture was subjected to ultrasonic treatment for 10 min to obtain a uniform mixture, the mixture was transferred to a 25 mL reaction flask, the flask was plunged with a rubber stopper, then vacuuming was carried out with a vacuum pump for 1 min, then a self-made hydrogen balloon was quickly plunged into to make the hydrogen pressure consistent with the atmospheric pressure, and stirred for 12 h at room temperature. Samples were taken every 1 h, extracted with an appropriate amount of ethyl acetate and centrifuged, the supernatant was taken, and the catalytic results were tested by gas chromatography-mass spectrometry (GC-MS).

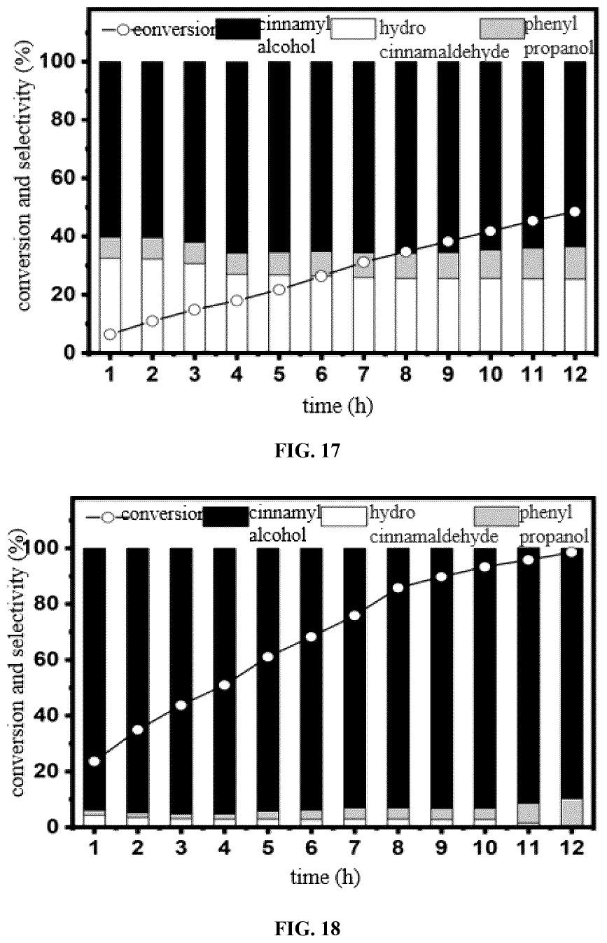

As shown in FIG. 17, when Pt/UiO-66-NH₂ is used as the catalyst, as the reaction time increases, cinnamaldehyde is continuously hydrogenated to produce cinnamyl alcohol, hydrocinnamaldehyde and phenylpropanol. After 12 h of reaction, the conversion of cinnamaldehyde is only 48.5%, the selectivity of cinnamyl alcohol in the product is 63.5%, and the performance is average.

As shown in FIG. 18, when PtFe₁/UiO-66-NH₂ is used as a catalyst, as the reaction time increases, cinnamaldehyde is continuously hydrogenated to produce cinnamyl alcohol, hydrocinnamaldehyde and phenylpropanol. After 12 h of reaction, the conversion of cinnamaldehyde is 98.5%, the selectivity of cinnamyl alcohol in the product is 89.6%, the reaction activity is much higher than that of pure Pt/UiO-66-NH₂, and it can produce cinnamyl alcohol with high selectivity.

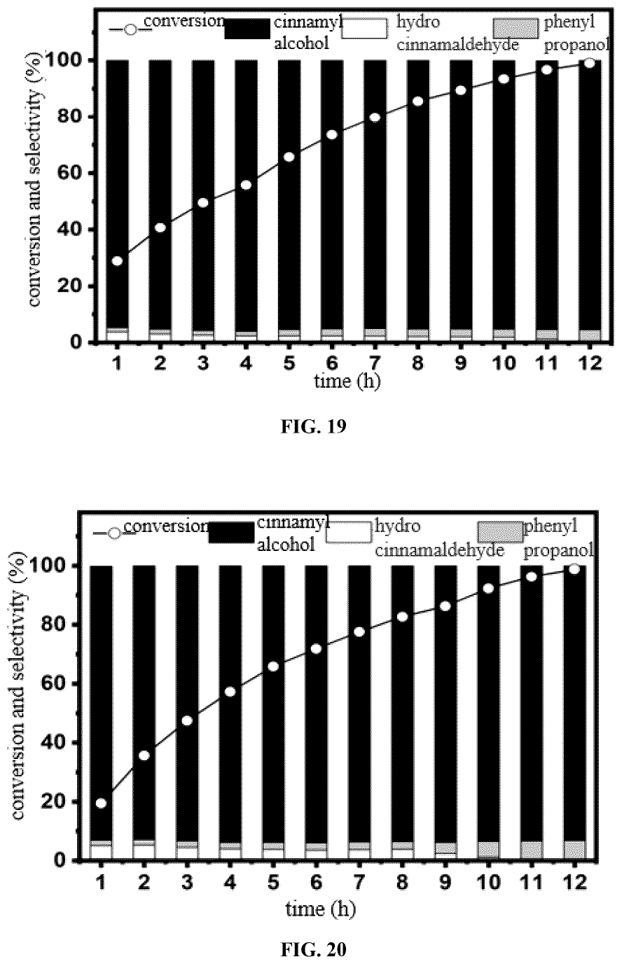

As shown in FIG. 19, when PtFe₂/UiO-66-NH₂ is used as a catalyst, as the reaction time increases, cinnamaldehyde is continuously hydrogenated to produce cinnamyl alcohol, hydrocinnamaldehyde and phenylpropanol. After 12 h of reaction, the conversion of cinnamaldehyde is 98.9%, the selectivity of cinnamyl alcohol in the product is 95.4%, the reaction activity is much higher than that of pure Pt/UiO-66-NH₂, and it can produce cinnamyl alcohol with high selectivity.

As shown in FIG. 20, when PtFe₃/UiO-66-NH₂ is used as a catalyst, as the reaction time increases, cinnamaldehyde is continuously hydrogenated to produce cinnamyl alcohol, hydrocinnamaldehyde and phenylpropanol. After 12 h of reaction, the conversion of cinnamaldehyde is 98.8%, the selectivity of cinnamyl alcohol in the product is 93.1%, the reaction activity is much higher than that of pure Pt/UiO-66-NH₂, and it can produce cinnamyl alcohol with high selectivity.

As shown in Table 2, PtCo₂/UiO-66-NH₂, PtNi₂/UiO-66-NH₂, PtFe₂/MIL-101 (Cr) and PtFe₂/UiO-66 also show good conversion and selectivity, the activity is better than that of pure Pt/UiO-66-NH₂ (the conversion and selectivity of Pt/UiO-66 are worse than Pt/UiO-66-NH₂).

From the above analysis, it can be seen that the PtFe/UiO-66-NH₂ with different amounts of Fe salt is better than the single metal catalyst Pt/UiO-66-NH₂ in terms of catalytic activity and product selectivity (PtFe₂/UiO-66-NH₂ performs best). After 12 h of reaction, the conversion of cinnamaldehyde hydrogenation catalyzed by PtFe/UiO-66-NH₂ is greater than 98%, and it can produce cinnamyl alcohol with selectivity greater than 90%, while the conversion catalyzed by Pt/UiO-66-NH₂ is less than 50%, it is also difficult to form a relatively single product, which proves that by adding Fe salt to form PtFe alloy and thin layer Fe-MOF, the conversion and product selectivity of the catalytic reaction can be greatly improved.

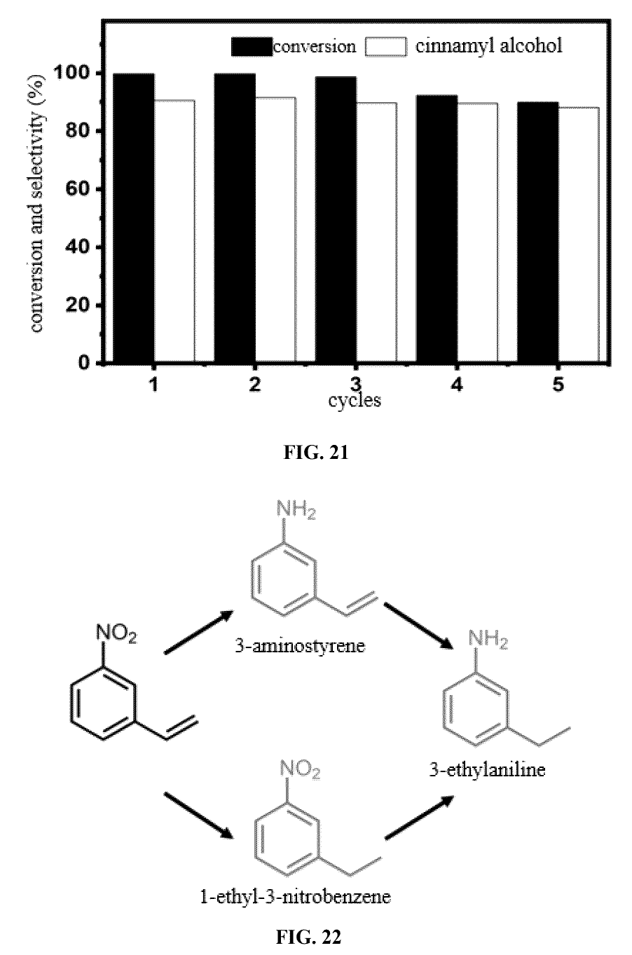

In addition, after 5 cycles of catalysis using PtFe₂/UiO-66-NH₂ (as shown in FIG. 21), its catalytic performance has not been significantly reduced, and it can still maintain high catalytic activity and product selectivity, which proves that the catalyst has good cycle stability, and further shows that the PtFe/UiO-66-NH₂ obtained in the present disclosure is an excellent catalyst for the selective hydrogenation of α, β-unsaturated aldehyde.

TABLE 2
Conversion and selectivity of various catalysts for catalytic hydrogenation of cinnamaldehyde for 12 h

| | | | | | selectivity | |
|---|---|---|---|---|---|---|
| | | | selectivity | selectivity | of | turnover |
| | | | of | of | cinnamyl | frequency/ |
| No. | catalyst | conversion/% | hydrocinnamaldehyde/% | phenylpropanol/% | alcohol/% | h⁻¹ |
| 1 | Pt/UiO-66 | 21.5 | 22.0 | 7.5 | 70.4 | 15.4 |
| 2 | Pt/UiO-66-NH₂ | 48.5 | 25.4 | 11.1 | 63.5 | 17.0 |
| 3 | PtFe₁/UiO-66-NH₂ | 98.5 | — | 10.4 | 89.6 | 36.4 |
| 4 | PtFe₂/UiO-66-NH₂ | 98.9 | 0.4 | 4.1 | 95.4 | 37.0 |
| 5 | PtFe₃/UiO-66-NH₂ | 98.8 | — | 6.8 | 93.1 | 41.4 |
| 6 | PtCO₂/UiO-66-NH₂ | 92.6 | 11.8 | 7.2 | 80.9 | 39.6 |
| 7 | PtNi₂/UiO-66-NH₂ | 98.0 | 3.7 | 70.8 | 25.5 | 36.2 |
| 8 | PtFe₂/MIL-101 (Cr) | 99.9 | 0.49 | 29.0 | 70.5 | 38.4 |
| 9 | PtFe₂/UiO-66 | 74.7 | 2.2 | 1.6 | 96.2 | 26.4 |
| 10 | UiO-66-NH₂ | N.D. | — | — | — | — |

### Experimental Example 4: Analysis of Activity and Selectivity of Selective Catalytic Hydrogenation of 3-Nitrostyrene

For the selective catalytic hydrogenation of 3-nitrostyrene, the catalytic ability and product selectivity of different catalysts were compared. The principle of the hydrogenation of 3-nitrostyrene is shown in FIG. 22. 3-Nitrostyrene can be hydrogenated to generate 3-aminostyrene or 1-ethyl-3-nitrobenzene under certain conditions, and then further hydrogenated to generate 3-ethylaniline. The ideal product is 3-aminostyrene.

The reaction conditions of heterogeneous catalytic hydrogenation are as follows: the catalyst and 3-nitrostyrene were added to 5 mL of ethanol with the added amount of catalyst of 5 mg and the added amount of 3-nitrostyrene of 0.3 mmol, the resulting mixture was subjected to ultrasonic treatment for 5 min to obtain a uniform mixture, the mixture was transferred to a 25 mL reaction flask, the flask was plunged with a rubber stopper, then vacuuming was carried out with a vacuum pump for 1 min, then a self-made hydrogen balloon was quickly plunged into to make the hydrogen pressure consistent with the atmospheric pressure, stirred for 5 h at room temperature, samples were taken every 1 h, and the catalytic results were tested by gas chromatography-mass spectrometry (GC-MS).

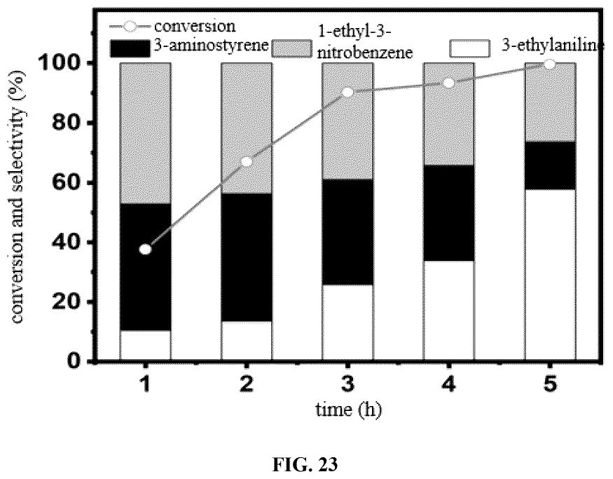

As shown in FIG. 23, when Pt/UiO-66-NH₂ is used as a catalyst, as the reaction time increases, cinnamaldehyde is continuously hydrogenated to produce 3-aminostyrene or 1-ethyl-3-nitrobenzene, and then further hydrogenated to generate 3-ethylaniline. After 3 h of reaction, the conversion of 3-nitrostyrene exceeds 90%, the selectivity of 3-aminostyrene in the product is 35.3%, and the product selectivity is not good.

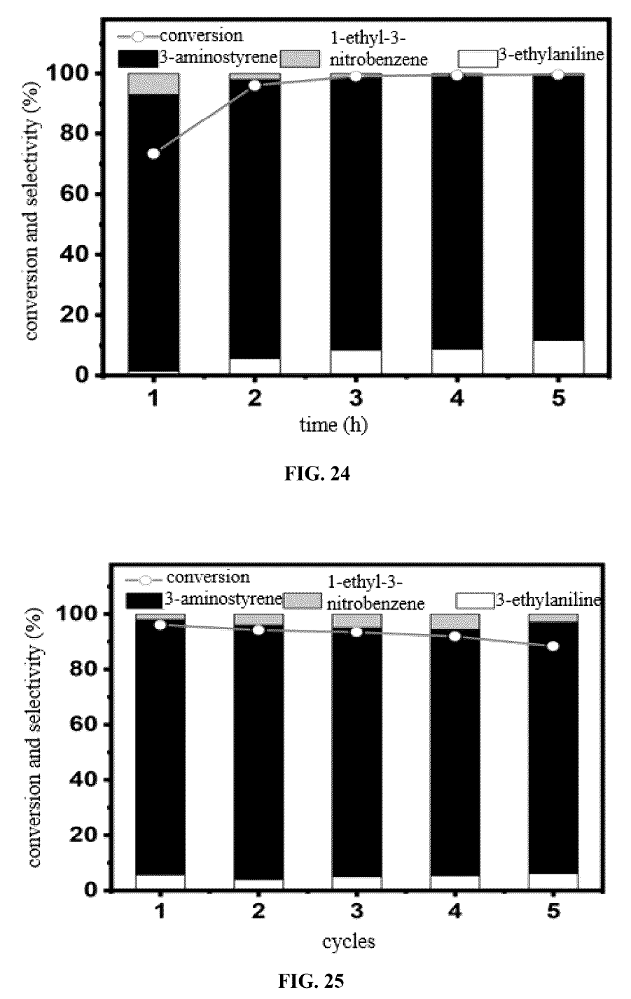

As shown in FIG. 24, when PtFe₂/UiO-66-NH₂ is used as a catalyst, as the reaction time increases, cinnamaldehyde is continuously hydrogenated to produce 3-aminostyrene or 1-ethyl-3-nitrobenzene, and then further hydrogenated to generate 3-ethylaniline. After 2 h of reaction, the conversion of 3-nitrostyrene reaches 96.1%, and the selectivity of 3-aminostyrene in the product is 92.3%, which proves that the catalyst can also be used for the selective hydrogenation of 3-nitrostyrene and obtain 3-aminostyrene with a high yield.

It can be seen from the above analysis that PtFe₂/UiO-66-NH₂ can well catalyze the hydrogenation of 3-nitrostyrene to obtain 3-aminostyrene, and prolonging the reaction time will not produce too much final product 3-ethylaniline, while pure Pt/UiO-66-NH₂ is far inferior to PtFe₂/UiO-66-NH₂ in terms of catalytic activity and product selectivity, which proves that the PtFe₂/UiO-66-NH₂ of the present disclosure has better performance in selective catalytic hydrogenation and has greater application value.

In addition, after 5 cycles of catalysis with PtFe₂/UiO-66-NH₂ (FIG. 25), it is found that the catalytic performance did not significantly decrease, which proves that the catalyst has good cycle stability. It can be seen that the catalyst of the present disclosure has high-efficiency selective hydrogenation performance and has good application prospects in hydrogen storage and hydrogenation and dehydrogenation.

### Experimental Example 5: Catalytic Dehydrogenation of Tetrahydroquinoline

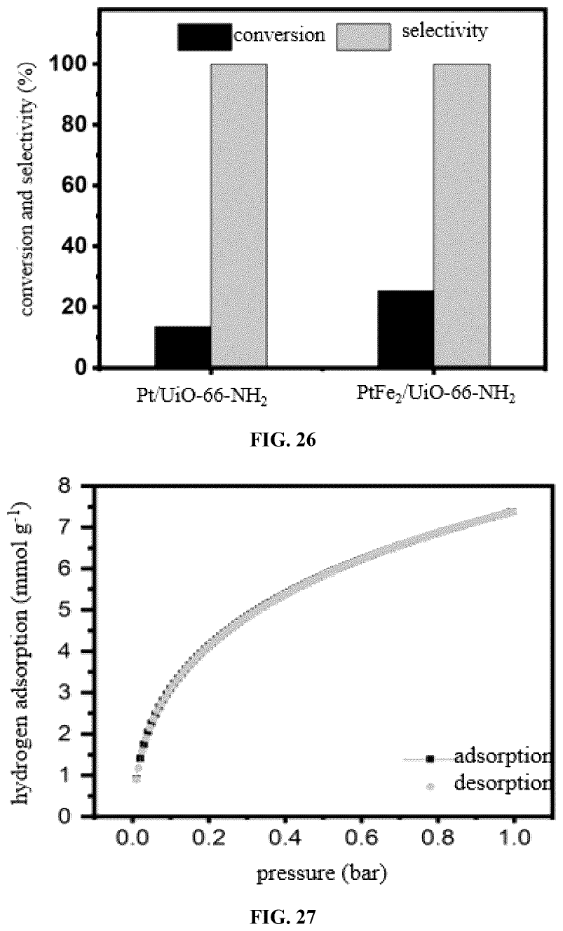

The conditions for the dehydrogenation of tetrahydroquinoline are as follows: 10 mg of Pt/MOFs catalyst and 0.1 mmol of tetrahydroquinoline were added to a mixture of 2 mL of xylene and 2 mL of water, the resulting mixture was subjected to ultrasonic treatment for 5 min, and heated to 100° C. to react for 20 h in the condensing reflux device and under stirring conditions. After the reaction, the mixture was transferred to a centrifuge tube and centrifuged, 100 μL of the supernatant was taken into a 1.5 mL sample bottle, 900 μL of ethanol was added thereto and mixed evenly. The catalytic results were tested by chromatography-mass spectrometer (GC-MS). The result is shown in FIG. 26. It can be seen from FIG. 26 that the Pt/MOFs catalyst prepared by the present disclosure has excellent catalytic performance for the dehydrogenation of tetrahydroquinoline to prepare quinoline.

### Experimental Example 6: Hydrogen Storage Capacity

The hydrogen storage capacity of 100 mg of PtFe₂/UiO-66-NH₂ was measured under the conditions of 1 bar and 77 K using a hydrogen adsorption device. The test results are shown in FIG. 27. It can be seen from FIG. 27 that the PtFe₂/UiO-66-NH₂ catalyst prepared by the present disclosure has excellent hydrogen storage performance under normal pressure.

The description of the above embodiments is only used to help understand the method and the core idea of the present disclosure. It should be pointed out that for those of ordinary skill in the art, without departing from the principle of the present disclosure, several improvements and modifications can be made to the present disclosure, and these improvements and modifications also fall within the protection scope of the claims of the present disclosure. Various modifications to these embodiments are obvious to those skilled in the art, and the general principles defined herein can be implemented in other embodiments without departing from the spirit or scope of the present disclosure. Therefore, the present disclosure will not be limited to the embodiments shown in this document, but should conform to the widest scope consistent with the principles and novel features disclosed in this document.

What is claimed is:

1. A method for preparing a Pt-based alloy/MOFs catalyst, wherein comprising the following steps: adding a MOFs carrier to DMF, dispersing and stirring at room temperature, then adding platinum acetylacetonate and terephthalic acid, continuing to stir at room temperature, adding a certain amount of acetylacetone metal salt, then stirring at room temperature, placing the resulting solution at 140-160° C. for continuous stirring for 10-15 h, and then performing centrifugation, washing and drying to obtain the Pt-based alloy/MOFs catalyst.

2. The method according to claim 1, wherein the MOFs carrier includes UiO-66-NH₂, UiO-66 or MIL-101(Cr).

3. The method according to claim 2, wherein the method for preparing UiO-66-NH₂ is as follows: adding zirconium tetrachloride and 2-aminoterephthalic acid to DMF, then adding acetic acid, stirring at room temperature, then reacting under high temperature of 110-130° C. and high pressure for 10-15 h, and finally performing centrifugation, washing and drying to obtain the UiO-66-NH₂.

4. The method according to claim 3, wherein in the preparation process of UiO-66-NH₂, the solid-to-liquid ratio of terephthalic acid to DMF is 36 mg/50 mL.

5. The method according to claim 3, wherein in the preparation process of UiO-66-NH₂, the solid-to-liquid ratio of zirconium tetrachloride to DMF is 52 mg/50 mL.

6. The method according to claim 3, wherein in the preparation process of UiO-66-NH₂, the volume ratio of acetic acid to DMF is 3:25.

7. The method according to claim 3, wherein in the preparation process of UiO-66-NH₂, the washing is washing twice with DMF and ethanol respectively; and the drying is vacuum drying at 120° C.

8. The method according to claim 1, wherein the acetylacetone metal salt includes ferrous acetylacetonate, nickel acetylacetonate, or cobalt acetylacetonate.

9. The method according to claim 1, wherein the solid-to-liquid ratio of the MOFs carrier to DMF is (40-60) mg/5 mL.

10. The method according to claim 1 or 9, wherein in the preparation process of the Pt-based alloy/MOFs catalyst, the ratio of the amount-of-substance of platinum acetylacetonate to the volume of DMF is 0.01 mmol:5 mL; and the solid-to-liquid ratio of terephthalic acid to DMF is (16-20) mg/5 mL.

11. The method according to claim 1, 8 or 9, wherein the ratio of the amount-of-substance of the acetylacetone metal salt to the volume of DMF is (0.01-0.03) mmol:5 mL.

12. The method of claim 1, wherein the Pt-based alloy/MOFs catalyst is washed twice with DMF and ethanol during the preparation process; and the drying is vacuum drying at 120° C.

13. A Pt-based alloy/MOFs catalyst prepared by the preparation method according to any one of claims 1-12.

14. The Pt-based alloy/MOFs catalyst according to claim 13, wherein the catalyst is PtFe₁/UiO-66-NH₂, PtFe₂/UiO-66-NH₂, PtFe₃/UiO-66-NH₂, PtM₂/UiO-66-NH₂, PtFe₂/UiO-66 or PtFe₂/MIL-101(Cr); where M in the PtM₂/UiO-66-NH₂ is Ni or Co.

15. Application of the Pt-based alloy/MOFs catalyst according to claim 13 or 14 in a selective hydrogenation reaction.

16. The application according to claim 15, wherein the selective hydrogenation reaction includes the selective hydrogenation reaction of α, β-unsaturated aldehyde and the selective hydrogenation reaction of 3-nitrostyrene.

17. The application according to claim 16, wherein the selective hydrogenation reaction of α, β-unsaturated aldehyde is a hydrogenation reaction of cinnamaldehyde.

18. The application according to claim 17, wherein the catalytic conditions for the hydrogenation reaction of cinnamaldehyde are as follows: adding the Pt-based alloy/MOFs catalyst and cinnamaldehyde to the mixture of 2.5 mL of water and 2.5 mL of isopropanol with the added amount of Pt-based alloy/MOFs catalyst of 5 mg and the added amount of cinnamaldehyde of 0.3 mmol, performing ultrasonic treatment for 10 min to obtain a uniform mixture, transferring the mixture to a 25 mL reaction flask, putting on a rubber stopper, performing vacuuming with a vacuum pump for 1 min, then quickly plunging into a self-made hydrogen balloon to make the hydrogen pressure consistent with the atmospheric pressure, and stirring for 12 h at room temperature.

19. The application according to claim 16, wherein the conditions for the selective hydrogenation of 3-nitrostyrene are as follows: adding the Pt-based alloy/MOFs catalyst and 3-nitrostyrene to 5 mL of ethanol with the added amount of Pt-based alloy/MOFs catalyst of 5 mg and the added amount of 3-nitrostyrene of 0.3 mmol, performing ultrasonic treatment for 5 min to obtain a uniform mixture, transferring the mixture to a 25 mL reaction flask, putting on a rubber stopper, performing vacuuming with a vacuum pump for 1 min, then quickly plunging into a self-made hydrogen balloon to make the hydrogen pressure consistent with the atmospheric pressure, and stirring for 5 h at room temperature.
